(19) United States
(12) Patent Application Publication
(10) Pub. No.: US 2009/0234284 A1
(43) Pub. Date: Sep. 17, 2009

(54) Safety Syringe Device

(76) Inventor: Wei-Shui WU, Taichung Hsien (TW)

Correspondence Address: HDLS Patent & Trademark Services, P.O. BOX 220746, CHANTILLY, VA, 20153-0746, US

(21) Appl. No.: 12/046,415

(22) Filed: Mar. 11, 2008

(51) Int. Cl. A61M 5/178

(52) U.S. Cl. 604/110

(57) ABSTRACT

A safety syringe device is composed of a syringe tube having a medicine compartment, a push rod penetrating through the medicine compartment, a needle seat screw engaging with the push rod at a top pusher where the push rod is emerged out of its interior threaded section, a needle base with a needle stem set on it is enclosed by a needle protector; wherein the syringe tube has an interior threaded section and a positioning screw thread formed at the interior of its front end. The needle seat is provided with an exterior threaded section around its outer circumference to screw combine with the positioning screw thread of the syringe tube, and with its guide flange to engage the needle protector which encloses the needle base with the interior threaded section such that the needle base is able to revolve on the needle seat towards its core bit and contact the core bit with its sloped surface thereby tightly setting the needle seat and needle base on the syringe tube.

### BACKGROUND OF THE INVENTION

1. Field of the Invention

The present invention relates to a safety syringe device, and more particularly, to a syringe device having a needle assembly capable of being retrieved into the syringe tube, and with better operational friction force and structural condition when its push rod and needle seat are combined, and having an easily fabricative structure.

2. Description of the Prior Art

Keeping in pace with level up of the scientific and medical technology, people have been more gradually aware of importance of good health, and pay more and more attention to the use of medical equipment. Improper treatment and disposal of used medical instruments, for example, the syringe needle intrusive to the human body is handled with less care, may cause severe injury to both the patient and medical personnel.

Nowadays, repeated use of the syringe needle and accidental puncturing of the skin with a used syringe needle during after-treatment are most problematic. Accordingly, the used syringe device must be thoroughly treat to an extent that it can never be used again, while during the treatment, the working personnel should be well protected not to be hurt by such a used syringe device. For solving these problems, the inventor of the present invention has contrived several safety syringe devices and actually acquired some of the related patents. In a bid to get better and simpler structure and attain optimal frictional force and compactness among component parts of the syringe device, he herein goes to great length of intensive research based on many years of experience acquired through professional engagement in the manufacturing of related products with continuous efforts for improvement finally come up with an improved structure of the present invention.

### SUMMARY OF THE INVENTION

The main object of the present invention is to provide a safety syringe device with a needle stem which is retrieval to the syringe tube after completion of injection by turning back to separate in the manner of breaking an exposed push rod at its neck where the diameter is reduced.

This safety syringe device with a simple, compact and easy to fabricate structure is composed of a syringe tube, a push rod, a needle seat, a needle stem and a needle base.

Inside the syringe tube there forms a medicine compartment provided with an interior threaded section and a positioning screw thread at its front end thereof, while a salient guillotine fringe is formed along the interior surface at the rear end.

The push rod which being inserted into the syringe tube has a top pusher of reduced diameter with a jut at its tip on the top portion thereof, and several billet like aligning clogs are disposed at a equal pitch around the lower exterior circumference of the top pusher. A guide surface sloping down to both sides is formed at the front end of each clog. Each clog has a ferrule underneath with a ring provided between the clog and the ferrule.

The front end of the needle seat is formed into a core bit of reduced diameter with a central path longitudinally passing through, and a stopper is provided in the central path, while the rear end of the needle seat is communicated with an accommodation cavity with several fixing blocks provided along the accommodation cavity, between two adjacent fixing blocks there is formed a barrier space. Around the exterior circumference of the accommodation cavity in the needle seat there are formed an exterior threaded section and a recessed annular groove. The exterior threaded section is to mate the interior threaded section formed in the syringe tube such that the needle seat can be tightly screw combined with the syringe tube.

By screw engaging the needle seat with the syringe tube, the needle stem can be set on the core bit, afterwards turn the positioning screw thread portion to tighten the whole structure, the safety syringe device is ready for operation by delivering the liquid drug from the medicine compartment in the syringe tube to the needle stem via the accommodation cavity and central path of the needle seat.

### BRIEF DESCRIPTION OF THE DRAWINGS

The drawings disclose illustrative embodiments of the present invention, which serve to exemplify the various advantages and objects hereof, and are as follow:

FIG. 1 shows an exploded view of the safety syringe device in a first embodiment of the present invention;

FIG. 2 is a sectional view of FIG. 1;

FIG. 3 is an illustrative view showing the push rod is ready in the position to be pushed form the rear end of the syringe tube to perform injection;

FIG. 4 is a three dimensional view of the push rod in a first embodiment;

FIG. 5 is a three dimensional view of the needle seat in a first embodiment;

FIG. 6 is a sectional view showing the push rod and needle stem are ready in the position to perform injection;

FIG. 7 is a sectional view showing the needle base is assembled on the needle seat for the needle stem ready to perform injection, and a partially enlarged view is provided for illustrating assembly manner;

FIG. 8 is the factionary perspective view of FIG. 6;

FIG. 9 is an illustrative view wherein the push rod is turned reversely to draw the needle seat downwards such that its exterior threaded section is liberated from the positioning screw thread of the syringe tube;

FIG. 10 is a schematic view showing the state that the needle seat and needle stem is remained in the syringe tube after breaking the neck portion of the push rod, and then the needle base is reversely inserted into the syringe tube where the needle stem penetrates through;

FIG. 11 is an exploded three dimensional view of the syringe tube in a second embodiment;

FIG. 12 is an assembly view of FIG. 11; and

FIG. 13 is a sectional view of the needle seat in a third embodiment assembled with the syringe tube shown in FIG. 11.

### DETAILED DESCRIPTION OF THE PREFERRED EMBODIMENT

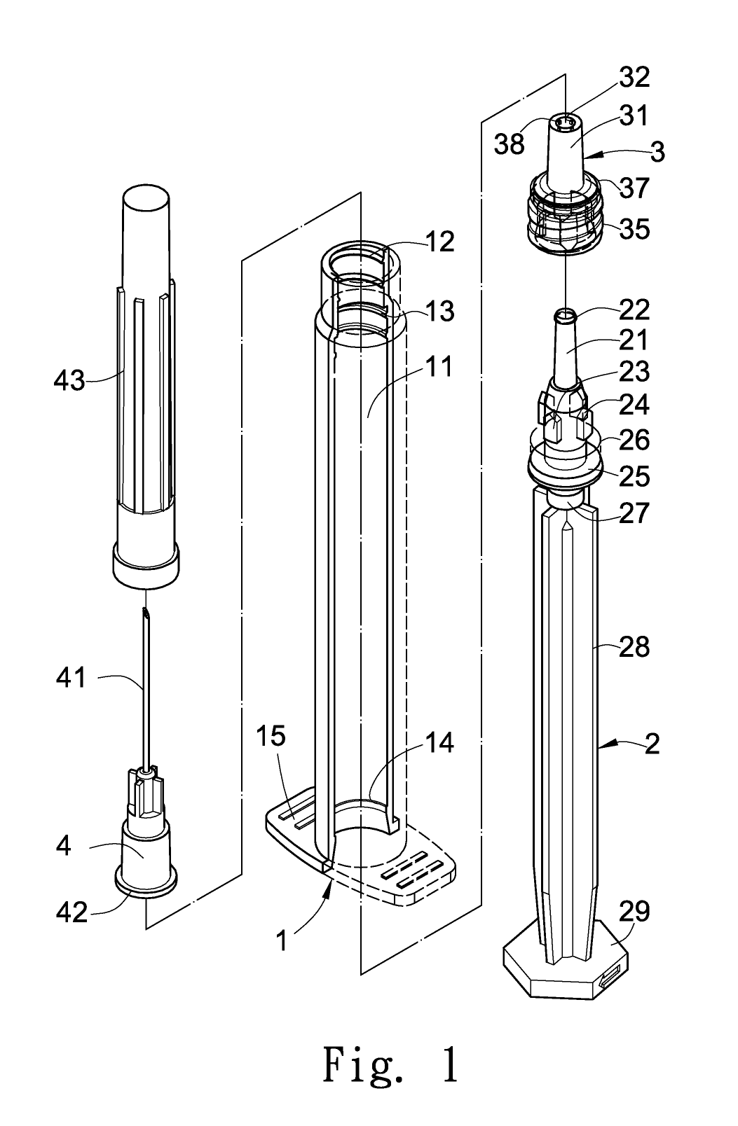

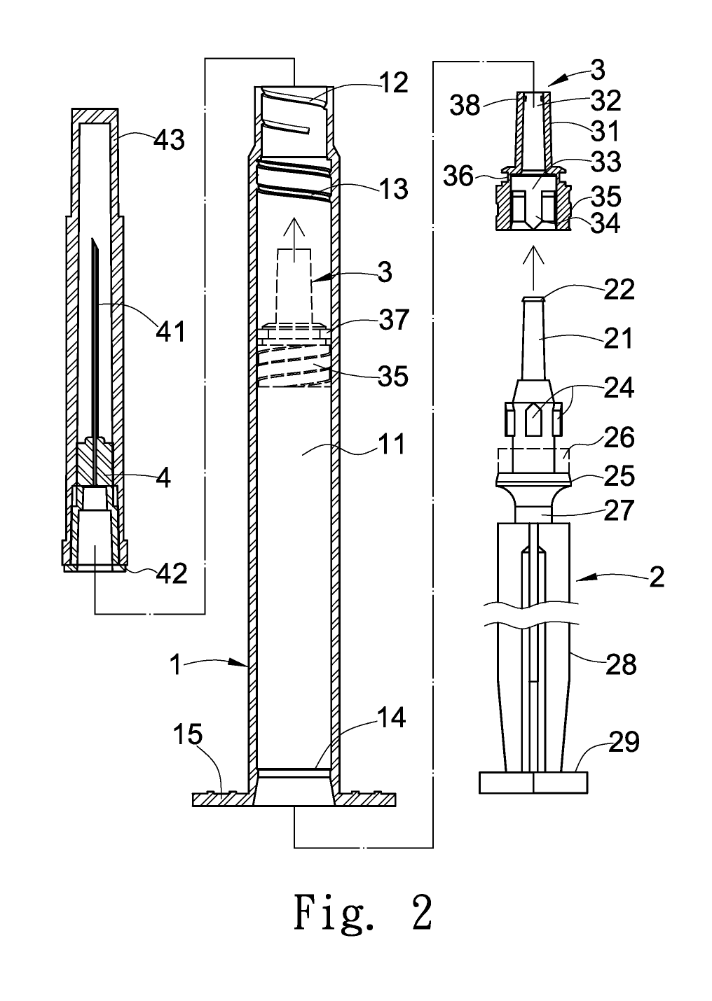

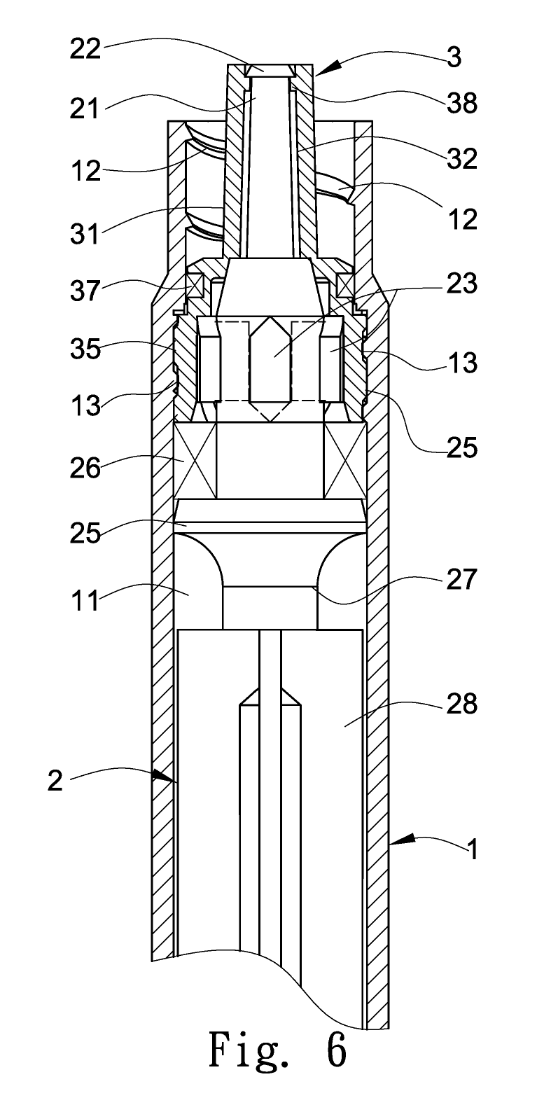

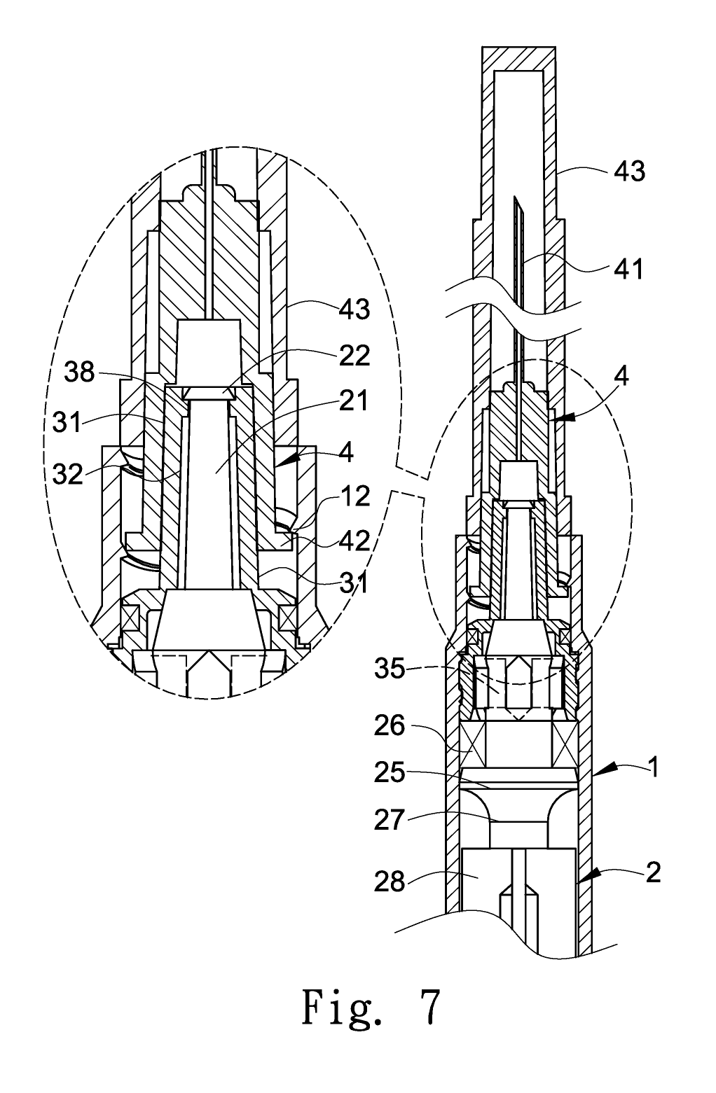

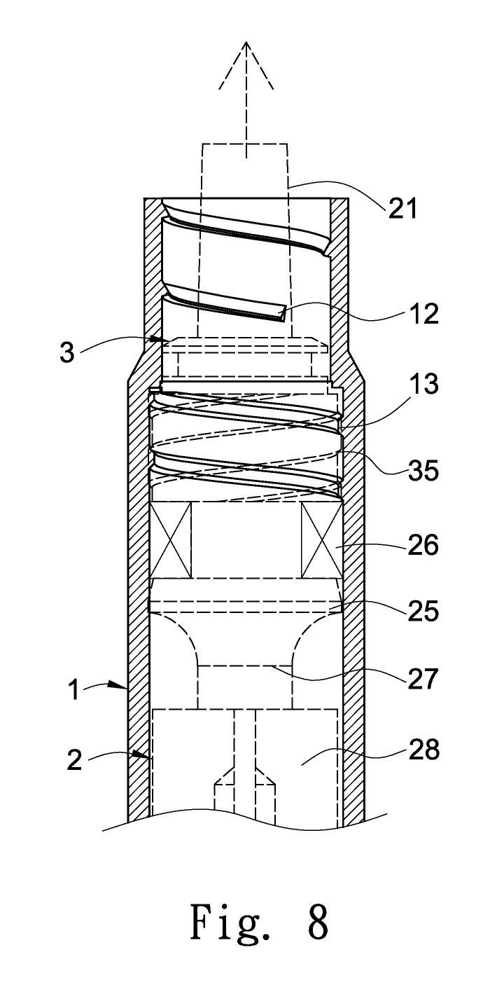

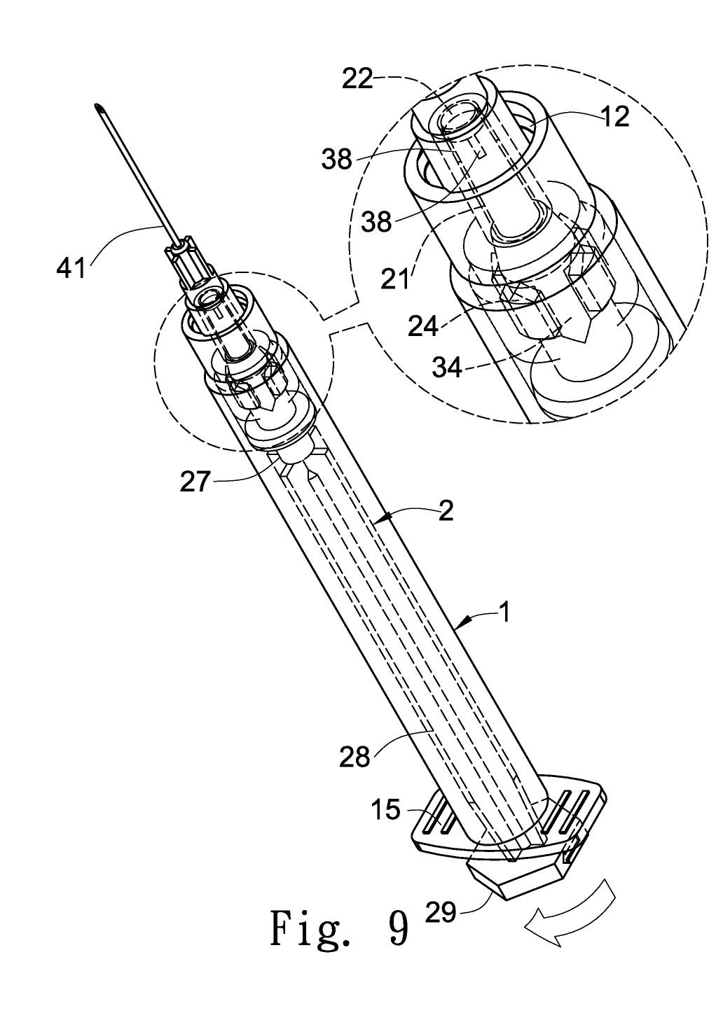

Referring to FIG. 1 through FIG. 8, the safety syringe device of the present invention comprises a syringe tube 1, a push rod 2, a needle seat 3, a needle base 4 and a needle stem 41.

The structure of the syringe tube 1 is substantially identical to those previously disclosed by the present inventor. It is essentially a cylindrical structure with a medicine compartment 11 formed inside at the center portion. At the front end inside the medicine compartment 11 there is a portion of reduced diameter having an interior threaded section 12 and a positioning screw thread 13, while at the rear end there is a salient guillotine fringe 14 formed along the interior surface thereof. At one end of the syringe tube 1 there is provided a pusher strip 15 to be pushed with the thumb.

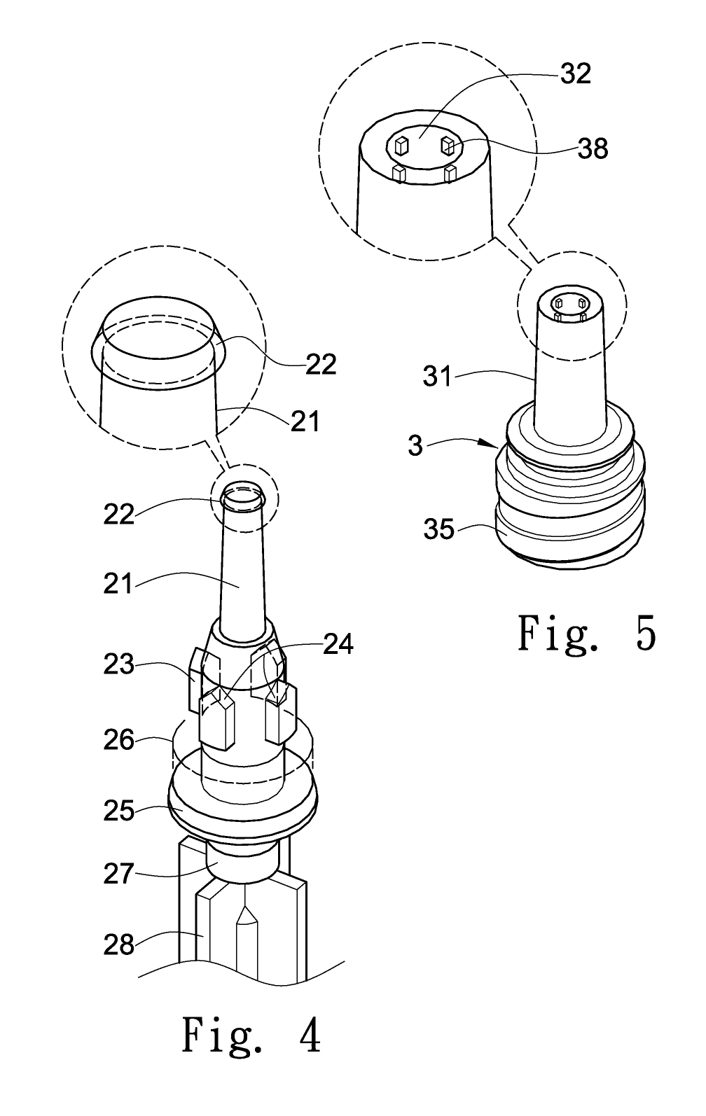

Referring again to FIG. 4 and FIG. 5, the push rod penetrating through the syringe tube 1 has a top pusher 21 of reduced diameter with a jut 22 at its top portion thereof, whereas the diameter of the jut 22 is slightly larger than that of the top pusher 21 on its top. Several billet like aligning clogs 23 are disposed at an equal pitch around the lower exterior circumference of the top pusher 21. A guide surface 24 sloping down to both sides is formed at the front end of each clog 23. Under the clog 23 there is a ferrule 25 with a ring 26 provided between the clog 23 and the ferrule 25. The ring 26 presses on the wall of the syringe tube 1 where the medicine compartment 11 is formed.

Under the ferrule 25 there is a neck portion 27 of reduced diameter in order to leave the section above it in the syringe tube 1 by twisting the push rod 2 after injection is over, and under the neck portion 27 there are several elongated ribs 28 whose terminal are attached with a pusher strip 29 to be pushed with thumb, an arrow head indication is marked at least on one side surface of the pusher strip 29.

The front end of the needle seat 3 is formed into a core bit 31 of reduced diameter with a central path 32 longitudinally passing through and a stopper 38 is provided in the central path 32, while at the rear end of the needle seat 3 there is an accommodation cavity 33 formed in communication with the central path 32. Several fixing blocks 24 are provided at an equal pitch around the surface of the accommodation cavity 33, between two adjacent fixing blocks 34 there is formed a barrier space. Around the exterior circumference of the accommodation cavity 33 in the needle seat 3 there are formed an exterior threaded section 35 and a recessed annular groove 36. The exterior threaded section 35 is to mate the interior threaded section 12 formed in the syringe tube 1 such that the needle seat 3 can be tightly screw combined with the syringe tube 1. A collar 37 is hooped around the recessed annular groove 36. Both threaded sections 35 and 12 employ square screw thread.

The needle stem 41 is inserted to or adhered to the needle base 4 and its bottom edge is formed into a guide flange 42, when the needle seat 3 comes to meet the interior threaded section 12 of the syringe tube 1, both the needle base 4 and the needle seat 3 can be set on the core bit 31 and fixed there, and the needle seat 3 and the needle stem 41 can be fixed by turning the positioning screw thread 13. by so, the medicine contained in the medicine compartment 11 in the syringe tube 1 can be delivered from the accommodation cavity 33 and flow through path 32 and needle stem 41 into the patient body. The bottom of the fixing block 34 is configurated into a sloping surface down to two sides from its center portion.

A conical needle protector 43 encloses the needle base 4 in the syringe tube 1 so as to avoid exposing the point of needle stem 41 that may accidentally puncture the human skin.

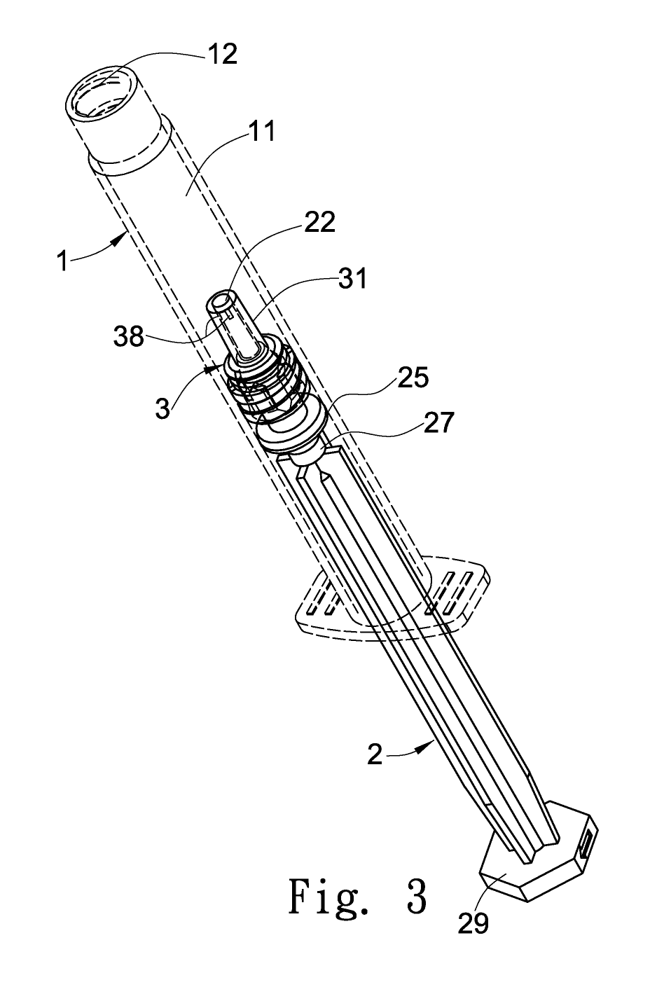

Referring to FIG. 3, when in using the safety syringe device constructed as such, at first put the needle seat 3 into the medicine compartment 11 of the syringe tube 1, and push the push rod 2 from the rear of the needle seat 3 to make its aligning clogs 23 trapped in the barrier spaces each formed between two adjacent fixing blocks 34 in the needle seat 3 thereby completing the assembly. Then continue to push the needle seat 3 onward with the push rod 2 until its core bit 31 reaches the positioning screw thread 13 of the syringe tube 1 to combine its exterior threaded section 35 with the positioning screw thread 13 such that the needle seat 3 can go onward by turning until its collar 37 is detained at the wall surface of the interior threaded section formed in the syringe tube 1. At this moment the ring 26 of the push rod 2 pressed around the inner wall surface of the syringe tube 1 so as to ensure the medicine compartment 11 perfectly hermetical. Afterwards needle base 4 with its needle protector 43, can be settled on the core bit 31 of the needle seat 3 by screw combining the guide flange 42 of the needle base 4 with the interior threaded section 12 such that the needle base 4 is able to revolve along the needle seat 3 and brings its sloped surface to fit on the core bit 31.

The operation to draw the medicine is identical to that of the traditional syringe device. After taking off the needle protector 43 and inserting the needle stem 41 into the medicine bottle (not shown), draw back the push rod 2 to absorb the medicine into the medicine compartment 11. This action can be reliably performed by reason that the needle seat 3 is firmly assembled with the syringe tube 1 by screw combining the exterior threaded section 35 and the positioning screw thread 13. After finishing injection, continue to push the push rod 2 forwards until the top end of the stopper 38 provided on the core bit 31 detains its jut 22.

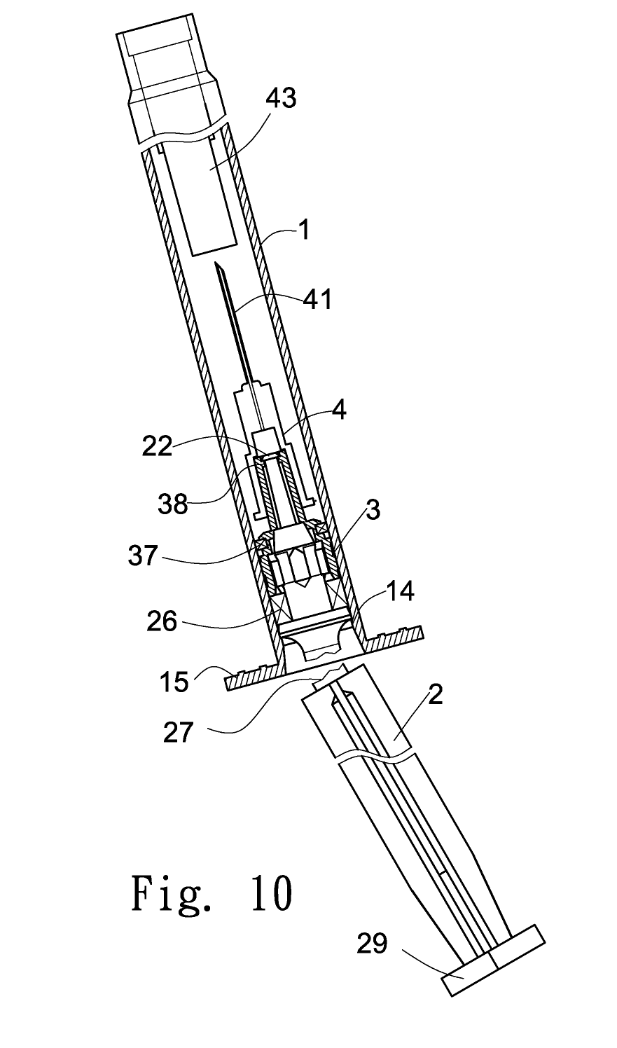

Afterwards turn the push rod 2 in the reverse direction to drive the needle seat 3 retreat along the helical route of the positioning screw thread 13 so as to liberate its exterior threaded section 35 out of the detention of the positioning screw thread 13,and also liberate the entire needle seat 3 out of the detention of the interior threaded section 12 of the syringe tube 1 thereby hiding the needle seat 3 together with the needle stem 41 in the medicine compartment 11 of the syringe tube 1. As soon as the neck portion 27 of the push rod 2 has retreated to face against the guillotine fringe 14 of the medicine compartment 11, the guillotine fringe 4 can sever the neck portion 27 by shaking the push rod 2; the needle stem 41 is left in the syringe tube 1 (see FIG. 10). After that reversely insert the needle protector 43 into the interior threaded section 12 and throw them into the disposal container.

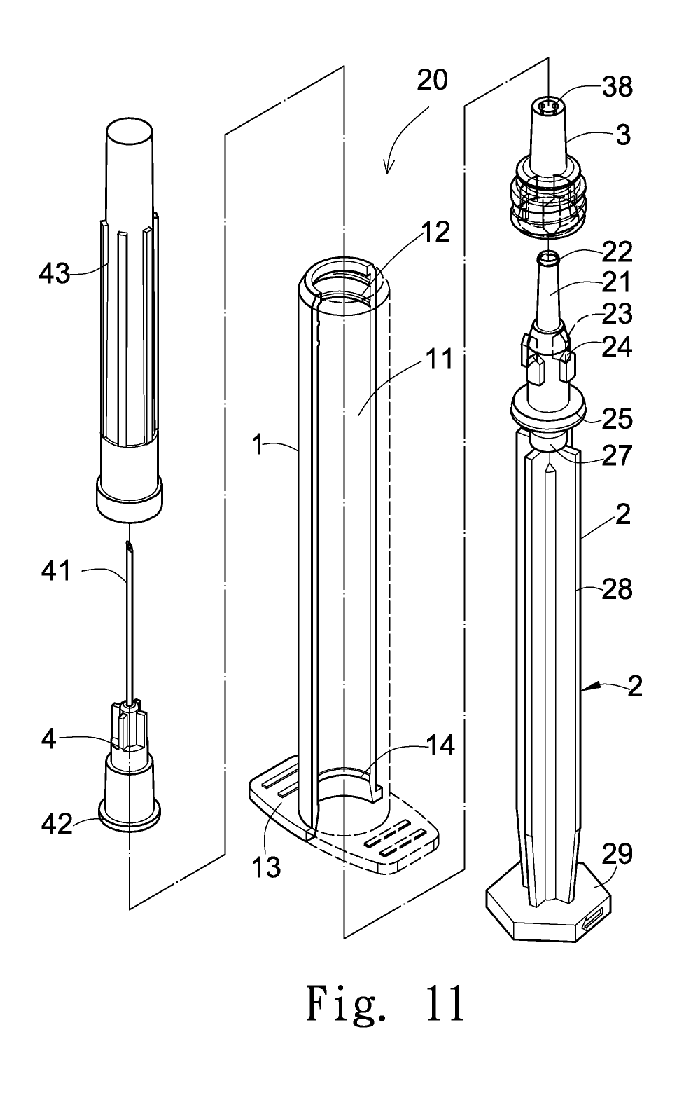

Referring to FIG. 11 and FIG. 12, in this second embodiment, the safety syringe device also comprises a syringe tube 1, a push rod 2 a needle seat 3, a needle stem 41 and a needle protector 43. The only difference from the first embodiment lies in that its syringe tube 1 only contains the positioning screw thread 13 but has no interior threaded section 12, however other structural and operational features remain the same.

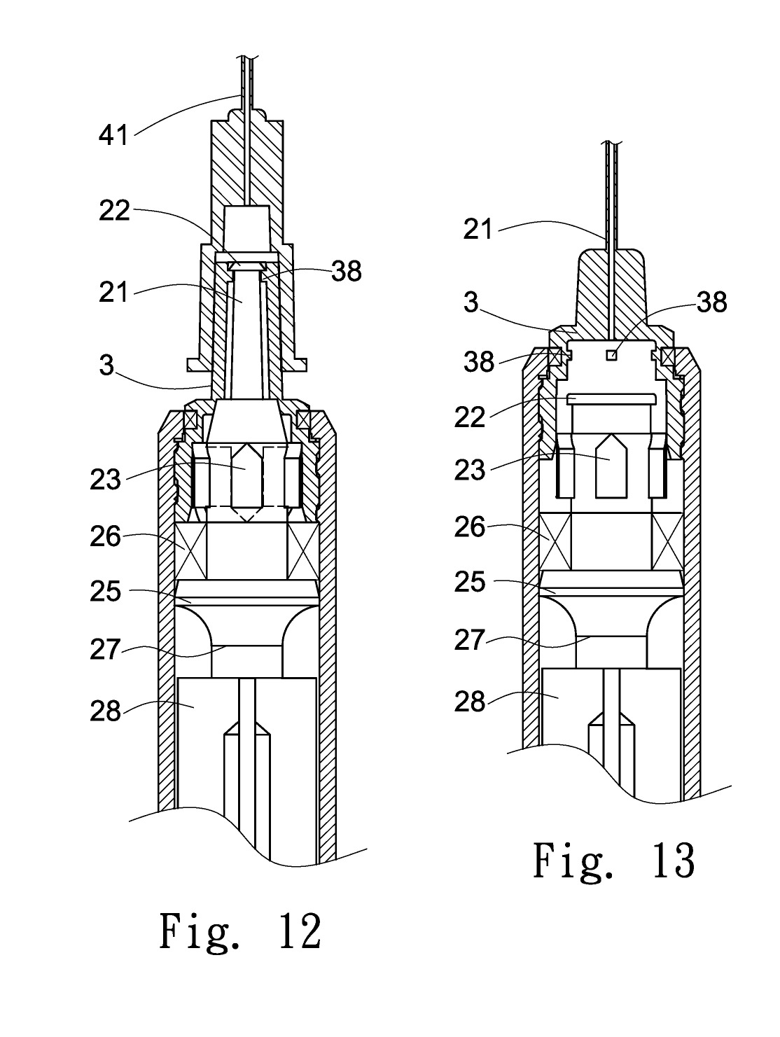

Referring to FIG. 13, in this third embodiment, similar to two previous embodiments, the safety syringe device comprises a syringe tube 1, a push rod 2, a needle seat 3, a needle stem 41 and a needle protector 43. The difference from the previous embodiments lies in that its syringe tube 1 only contains the positional screw thread 13 but has no interior threaded section 12, and the push rod 2 is not equipped with the top pusher 21, it has only the jut 22. The needle stem 41 is directly affixed to one end of the needle seat 3 without employing the core bit 31 and path 32. Around the surface of the accommodation cavity 33 of the needle seat 3, there are provided several fixing blocks 34 disposed at an equal pitch and a stopper 38. Other structural and operational features of the component parts push rod 2, needle seat 3, needle stem 41 and needle protector 43 remain the same as the previous embodiments.

It emerges from the description of the above embodiments that the invention has several noteworthy advantages, in particular:

(1) The jut 22 of the push rod 2 can be detained by the stopper 38 of the core bit 31 that enables the medicine in the syringe tube 1 to be completely injected into the patient body without wasting.

(2) The used needle stem 41 is restored in the syringe tube 1 without the worry of exposing to puncture anyone's skin. All used component parts including syringe tube 1, push rod 2, needle seat 3 and needle stem 41 can not be restored for reuse thereby the medical security is assured.

(3) The needle seat 3 and the syringe tube 1 are engaged with each other with square screw threaded sections 35 and 12 that ensures reliable structural security and improves yield of fabrication of the syringe device.

(4) The provision of fixing blocks 34 with intermediate barrier spaces to confine the aligning clogs 23 of the push rod 2 contributes to supplying an appropriate frictional force when operating the device. The ring 26 of the push rod 2 performs the function of effectively maintaining perfect hermetical state in the medicine compartment 11 without the fear of leaking medicine.

Those who are skilled in the art will ready perceive how to modify the invention. Therefore the appended claims are to be construed to cover all equivalent structures, which fall within the true scope and spirit of the invention.

What is claimed is:

1. A safety syringe device comprising:
a syringe tube with a medicine compartment which is provided with an interior threaded section and a positioning screw thread at its front end in order;
a push rod penetrating through said syringe tube and having a top pusher with a jut on its top; wherein the diameter of said jut is slightly larger than that of said top pusher on its top, several aligning clogs are disposed around the lower exterior circumference of said top pusher, and a ferrule is formed immediately below, and beneath said ferrule there is formed a neck portion of reduced diameter;
a needle seat having a core bit of reduced diameter at the front end thereof, a central path being longitudinally passing through it and a stopper being provided therein, and an accommodation cavity formed at the rear end of said needle seat, and several fixing blocks provided around the surface of said accommodation cavity, between said two adjacent fixing blocks there being formed a barrier space to trap the aligning clog of said push rod, an exterior threaded section being formed around the exterior circumference of said accommodation cavity to screw engage with the positioning screw thread of said syringe tube; and
a needle base having a needle stem provided at one end thereof, while a guide flange formed at the other end thereof to screw engage with the interior threaded section of said syringe tube such that said needle base is able to revolve along said needle seat and brings its sloped surface to fit on said core bit.

2. The safety syringe device of claim 1, wherein said aligning clog of said push rod has a guide surface at its front end sloping down to two sides from the center.

3. The safety syringe device of claim 1, wherein the bottom of said fixing block of said needle seat is configurated into a sloping surface down to two sides from its center portion.

4. A safety syringe device comprising:
a syringe tube with a medicine compartment which is provided with an inner positioning screw thread at the front end thereof,
a push rod penetrating through said syringe tube and having a top pusher with a jut on its top; wherein the diameter of said jut is slightly larger than that of said top pusher on its top, several aligning clogs are disposed around the lower exterior circumference of said top pusher, and a ferrule is formed immediately below, and beneath said ferrule there is formed a neck portion of reduced diameter; and
a needle seat having a core bit of reduced diameter at the front end thereof, a central path being longitudinally passing through it and a stopper being provided therein, and an accommodation cavity formed at the rear end of said needle seat, and several fixing blocks provided around the surface of said accommodation cavity, between said two adjacent fixing blocks there being formed a barrier space to trap the aligning clog of said push rod, an exterior threaded section being formed around the exterior circumference of said accommodation cavity to screw engage with the positioning screw thread of said syringe tube.

5. The safety syringe device of claim 4, wherein said aligning clog of said push rod has a guide surface at its front end sloping down to two sides from the center.

6. The safety syringe device of claim 4, wherein the bottom of said fixing block of said needle seat is configurated into a sloping surface down to two sides from its center portion.

7. The safety syringe device of claim 4, said syringe device further comprises a needle protector set on said needle base to enclose and protect said needle base and said needle stem.

8. The safety syringe device of claim 4, wherein after being finished injection, said push rod is continued to be pushed forwards until said jut is detained by the top end of a stopper provided on said core bit, afterwards turning said push rod in the reverse direction to drive said needle seat retreat along the helical route of said positioning screw thread so as to liberate its exterior threaded section out of the detention of said positioning screw thread, and also liberate the entire needle seat pout of the detention of said interior threaded section of said syringe tube.

9. A safety syringe device comprising:
a syringe tube with a medicine compartment which is provided with a positioning screw thread interior of its front end;
a push rod penetrating through said syringe tube and having a jut on the top end thereof, several aligning clogs being disposed around its exterior circumference beneath said jut, and a ferrule being formed immediately below said clogs; and
a needle seat with a needle stem directly adhered to one end thereof, and an accommodation cavity connected to its path, several fixing blocks and a stopper being provided around the surface of said accommodation cavity, between two adjacent fixing blocks there being formed a barrier space to trap said clog of said push rod, and an exterior threaded section being formed around the external circumference of said accommodation cavity to screw engage with said positioning screw thread of said syringe tube.
